(19) United States
(12) Patent Application Publication
(10) Pub. No.: US 2004/0101503 A1
(43) Pub. Date: May 27, 2004

(54) Use of protectin activator to enhance the skin's resistance, composition comprising such activators and selection method

(75) Inventors: Yann Mahe, Morsang Sur Orge (FR); Alain Meybeck, Courbevoie (FR)

Correspondence Address: BURNS DOANE SWECKER & MATHIS L L P, POST OFFICE BOX 1404, ALEXANDRIA, VA, 22313-1404, US

(73) Assignee: SOCIETE L'OREAL S.A., Paris (FR)

(21) Appl. No.: 10/656,296

(22) Filed: Sep. 8, 2003

Related U.S. Application Data
(60) Provisional application No. 60/418,234, filed on Oct. 15, 2002.

(30) Foreign Application Priority Data
Sep. 6, 2002 (FR) 02/11078

(51) Int. Cl. A61K 7/06; A61K 7/11

(52) U.S. Cl. 424/70.14

(57) ABSTRACT

The invention relates to the use of at least one protectin or protectin precursor production activator in a cosmetic composition or for preparing a composition intended to limit the side-effects of complement activation on the skin, mucosae and/or scalp. It also relates to compositions comprising such activators, to a method of selecting a product capable of protecting the cell membranes and a cosmetic method of enhancing the skin's resistance to the effects of complement activation.

### CROSS-REFERENCE TO PRIORITY/PROVISIONAL APPLICATIONS

This application claims priority under 35 U.S.C. § 119 of FR-02/11078, filed Sep. 6, 2002, and of provisional application Serial No. 60/418,234, filed Oct. 15, 2002, both hereby expressly incorporated by reference. This application is also a continuation of said '234 provisional.

### BACKGROUND OF THE INVENTION

1. Technical Field of the Invention

The present invention relates to the use of substances which strengthen the skin's resistance by activating the production of protectin or protectin precursor by skin cells, in particular in cosmetic or dermatological compositions. It also relates to compositions which comprise such substances and to a method of selecting compounds which can be used in these compositions.

2. Description of Background/Related/Prior Art

The skin constitutes a barrier against aggressive external influences, especially chemical, mechanical or infectious influences, and consequently a certain number of defense reactions against environmental factors (climate, ultraviolet radiation, tobacco, etc.) and/or against xenobiotics, such as microorganisms, for example, take place at the skin. In a healthy person, these defense mechanisms are effective at preventing the spread of infection.

These defense mechanisms, however, although beneficial to the organism, can give rise to locally undesirable reactions, which may consist in a simple discomfort or change in appearance, considered unattractive by the person sensing it. In other cases, these local reactions may be excessive and may themselves give rise to maladjustments of physiological functioning.

It would therefore be desirable to have actives which would protect the skin from the undesirable effects of defense reactions by the organism without, however, hampering the functioning of these mechanisms, which are indispensable for protecting the organism against external agents.

The skin is composed of two compartments, one on the surface, the epidermis, and one lower down, the dermis, which interact. Natural human epidermis is composed principally of three types of cell, these being keratinocytes (the vast majority), melanocytes and Langerhans' cells. Each of these cell types contributes, through its specific functions, to the essential role played by the skin in the body, particularly the role of protecting the body from external aggressive influences, which is referred to as the “barrier function”.

The epidermis is conventionally divided into a basal layer of keratinocytes, which constitutes the germinative layer of the epidermis, a so-called prickle cell layer consisting of a number of layers of polyhedral cells arranged on the germinative layers, from one to three layers known as granular layers, which consist of flattened cells containing distinct cytoplasmic inclusions, the keratohyalin granules, and finally the horny layer (or stratum corneum), which consists of a collection of layers of keratinocytes at the final stage of their differentiation, referred to as corneocytes.

The dermis gives the epidermis a solid support. It is also its nutritional element. It consists principally of fibroblasts and of an extracellular matrix composed primarily of collagen, elastin and a substance called ground substance. These components are synthesized by fibroblasts. Also present therein are leukocytes, mastocytes or else tissue macrophages. Finally, the dermis is traversed by blood vessels and nerve fibers.

Cohesion between the epidermis and the dermis is ensured by the dermal-epidermal junction. This is a complex region with a thickness of approximately 100 nm which comprises the basal pole of the basal keratinocytes, the epidermal membrane and the sub-basal region of the superficial dermis.

Protection against external factors involves in particular the activation of the complement, by a specific pathway of an antibody or by an alternative pathway constituting a means of immediate defense. The complement consists of twenty circulating proteins whose activation in cascade is the origin of the appearance of various biological activities, such as cellular, bacterial or viral lysis. In physiological situations it contributes in particular to bacterial protection and to wound coagulation. Its activation is rapid and localized. Complement activation contributes significantly, however, to the pathogenesis of inflammatory states and to certain autoimmune reaction mechanisms.

The origin of these phenomena is in particular the attack of cell membranes by the complex formed by complement activation, when its activation is turned against the attacked organism's own cell constituents.

### SUMMARY OF THE INVENTION

Unexpectedly the applicant has now found that certain compounds are capable of activating specifically the endogenous production of a protein which allows the cells, especially the skin cells, to be protected against these attacks, and does so without lessening the defenses of the organism against external agents.

### DETAILED DESCRIPTION OF BEST MODE AND SPECIFIC/PREFERRED EMBODIMENTS OF THE INVENTION

The present invention accordingly provides for the use of at least one modulator of protectin or protectin precursor production, in particular a protectin or protectin precursor production activator, in a cosmetic composition or for preparing a composition the modulator or the composition being intended for enhancing the resistance of the skin, mucosae and/or scalp to the non-specific side-effects of complement activation. Advantageously the composition is intended for application topically to the skin, mucosae and/or scalp. In exemplary embodiments, the modulator and/or composition is administered in an amount effective to enhance the resistance of the skin to such non-specific side-effects of complement activation.

Protectin, also called CD59, is a glycoprotein naturally present in the skin, which forms part of the physiological regulators of the complement. Its structure has in particular been described by Sawada et al. (DNA Cell Biol., 1990, 9(3): 213-20). Its functions include, especially, that of downwardly regulating the formation of the cell membrane attack complex (Venneker et al., Exp. Clin. Immunogenet., 1992, 33-47).

The compositions according to the invention will strengthen the resistance of the skin, mucosae or hair follicles to the side-effects of complement activation and will therefore improve its tolerance. The compounds which modulate, and in particular stimulate, the endogenous production of protectin or protectin precursor, or the compositions comprising them, are therefore intended to limit the non-specific cutaneous effects of complement activation. The compounds or compositions in question will in particular be actives with a soothing and/or anti-irritant effect. They therefore reduce the appearance of redness or of small unsightly spots.

The compositions and/or the activators of endogenous production of protectin or protectin precursor in accordance with the invention are thus useful in preventing, reducing or treating erythema, especially solar erythema. The role of the complement in reactions accompanying excessive exposure of the skin to UV radiation, in particular UVB, has in fact been demonstrated, and protecting the cell membranes against the complexes resulting from its activation will reduce its effects associated with skin hyperreactivity.

In accordance with another aspect of the invention the activator or composition comprising it is intended to treat skins suffering from acne or having a tendency towards acne. More particularly the activator or composition will be intended to care for pre-acne skin and/or seborrhoeic skin in order to prevent or attenuate the redness associated with the irritation of the hair follicle.

The activators of endogenous production of protectin or protectin precursors will be useful in accordance with the invention for reducing the signs of rosacea and, generally, the unattractive signs of hair weakness, such as the appearance of localized redness in cases of repeated rubbing, associated for example with repeated blowing of the nose in case of cold.

In accordance with yet another of the aspects of the invention, the compositions or activators of endogenous production of protectin or protectin precursor are intended to treat sensitive skin.

Subjects said to have a sensitive skin or scalp constitute a group which has now been well identified. They are characterized by a collection of symptoms which are, especially, subjective signs such as unpleasant sensations. By unpleasant sensations are meant more or less painful sensations felt within a skin area, such as stinging, tingling, itching or pruritus, burning, discomfort, tautness, etc. The sensitive skins can be divided into two major clinical forms: irritable and/or reactive skin, and intolerant skin. The other signs which may be associated with the unpleasant sensations are redness or erythema, sores and, within the subclass of intolerant skins, a hyperseborrhoea. The essential characteristic of sensitive skin is a mechanism of response to external factors which may affect any individual, even if individuals said to have sensitive skin react more rapidly to these factors than do others. This mechanism, however, is not immunological; it is aspecific.

“Sensitive” scalps have a less ambiguous clinical semiology: the sensations of pruritus and/or of stinging and/or of burning are essentially triggered by local factors such as rubbing, soap, surfactants, hard water with a high calcium concentration, shampoos or lotions. These sensations are also sometimes triggered by factors such as the environment, emotions and/or food. Erythema and hyperseborrhoea of the scalp, and a hair condition, are frequently associated with the aforementioned signs.

In order to determine whether skin is sensitive or not, a capsaicin test can be carried out, as described in particular in EP-723,774.

A sensitive skin is therefore not an allergic skin. An allergic skin is in fact a skin which reacts to an external agent, an allergen, which triggers an allergic reaction. This is an immunological process which actually involves the pathway of activation of the complement, which takes place only when an allergen is present and which affects only sensitized subjects. According to another of the aspects of the invention, the compositions or activators of endogenous production of protectin or of a protectin precursor are intended, while not eliminating the allergic reaction, to limit the harmful consequences thereof within the skin tissue by preventing the proteins of the complement from destroying the cell membranes of the infiltrated tissue.

In accordance with one version of the invention the protectin or protectin precursor production activators and/or compositions will be used to prevent and/or reduce the appearance of grey or white hairs in human beings, which is referred to as canities. This is because it is known that, in the whitening of the hair, the melanocytes are destroyed by the immune system. The use of a protectin or protectin precursor production activator will aid the melanocytes to protect themselves against this attack.

In certain situations, indeed, it has been shown that an inflammatory component including the perifollicular infiltrates can hamper the emergence of the hair, and may even be responsible for its loss. In these situations, referred to as alopecic situations, the activator or composition will be intended to care for scalps characterized by an abnormally high hair loss, which may cause baldness.

In accordance with another version of the invention, the protectin or protectin precursor production activators and/or compositions will be used to prevent and/or reduce the loss of hair, and/or to enhance hair density, by reducing the activity of the complement cascade, responsible in part for the stiffening of the follicle sheath (Mahé et al., Int. J. Dermatol.).

According to another embodiment, the modulator of protectin production is used in compositions intended for the treatment of vitiligo, in particular as back-up treatment and/or to reduce some of the signs associated to vitiligo.

The invention also provides the use of at least one protectin or protectin precursor production activator for preparing a composition intended for application to the skin, mucosae and/or scalp, the said composition and/or activator being intended to treat at least one disorder and/or at least one sign associated with a disorder selected from herpes, psoriasis, allergy, seborrhoeic dermatitis, disorders caused by Pityrosporum ovale or Malassezia, especially Malassezia furfur, chronic skin ulcers, urticaria and immunological diseases, particularly maladjustments of the complement activation system. More specifically the activator or the composition comprising it is intended to prevent or reduce the skin signs associated with these disorders, in particular with allergic skin.

According to another embodiment, the modulator of protectin production can be used in compositions intended for the treatment of vitiligo, in particular as back-up treatment and/or to reduce some of the signs and/or symptoms associated with vitiligo.

The compositions or the activators according to the invention will be used advantageously in subjects suffering from herpes, as a back-up treatment between two eruptive episodes, these eruptive episodes normally being manifested in a reaction commonly referred to as “fever spot”.

The invention hence likewise provides cosmetic, dermatocosmetic or dermatological compositions comprising at least one activator of endogenous production of protectin or a protectin precursor.

The protectin or protectin precursor production activator may be any substance or mixture of substances capable of stimulating the synthesis of this glycoprotein by cells, in particular by skin cells. It may in particular comprise extracts of single-celled or multicellular organisms, such as complex or purified plant extracts, or of chemically defined compounds, and also mixtures of these.

The protectin or protectin precursor production activator will advantageously be selected from the group consisting of bacterial extracts, especially extracts of non-photosynthetic filamentous bacteria, lactic bacteria and their extracts, in particular from the genera Lactobacillus or Bifidibacterium, ginseng extracts, especially saponins such as ginsenosides, particularly the ginsenosides Rb1 and Rg1, derivatives thereof and mixtures thereof.

Preferably, if the activator is a total extract of Vitreoscilla filiformis, it is combined with at least one other protectin or protectin precursor production activator. This is because extracts of Vitreoscilla filiformis are particularly useful for stimulating endogenous production of protectin by keratinocytes. They will advantageously be combined with compounds of stimulating the endogenous production of protectin or a protectin precursor either by keratinocytes or by other types of skin cells, in particular by fibroblasts. The composition advantageously comprises at least one keratinocyte protectin or protectin precursor production activator in combination with at least one fibroblast protectin or protectin precursor production activator. In particular the keratinocyte protectin or protectin precursor production activator is selected from extracts of non-photosynthetic filamentous bacteria and the fibroblast protectin or protectin precursor production activator is selected from the group consisting of ginseng extracts and ginsenosides.

The term “extract of non-photosynthetic filamentous bacteria” refers equally to the culture supernatant of the said bacteria, the biomass obtained after culturing the said bacteria or else the biomass extracts obtained by treating this biomass.

The bacterial extracts according to the invention are prepared from non-photosynthetic filamentous bacteria as defined in accordance with the classification of Bergey's Manual of Systematic Bacteriology (vol. 3, sections 22 and 23, 9th edition, 1989), among which mention may be made of the bacteria belonging to the order of the Beggiatoales, and more particularly their bacteria belonging to the genera Beggiatoa, Vitreoscilla, Flexithrix or Leucothrix.

The bacteria which have just been defined, and of which a number have already been described, generally have an aquatic habitat and may be found in particular in seawater or spring water. Among the bacteria which can be used mention may be made, for example, of the following:

Vitreoscilla filiformis (ATCC 15551)

Vitreoscilla beggiatoides (ATCC 43181)

Beggiatoa alba (ATCC 33555)

Flexithrix dorotheae (ATCC 23163)

Leucothrix mucor (ATCC 25107)

Sphaerotilus natans (ATCC 13338)

In order to prepare the extract according to the invention the said bacteria can be cultivated in accordance with the methods known to the person skilled in the art, then separated from the resultant biomass, by filtration, centrifugation, coagulation and/or lyophilization, for example.

The extracts which can be used in accordance with the invention may be prepared in particular by the process described by the applicant in WO-A-93/00741.

Thus, after culturing, the bacteria are concentrated by centrifugation. The biomass obtained is autoclaved. This biomass can be lyophilized to give what is referred to as the lyophilized extract. Any method of lyophilization known to the person skilled in the art can be used to prepare this extract.

The supernatant fraction of this biomass may also be filtered in a sterile container in order to remove the suspended particles. This gives the extract which is referred to elsewhere in the text as aqueous extract.

EP-604,631 describes extracts of filamentous bacteria as being an agent which stimulates non-specific immunity. However, unexpectedly, the applicant has now shown that extracts of such bacteria may be used as agents which stimulate the endogenous synthesis of protectin or protectin precursor. According to other embodiments of the invention, the extracts described in EP-765,567 can be used.

By ginseng is meant here the plant Panax ginseng, and also plants of the same family such as Panax notoginseng (San Qi), Panax quinquefolium or Panax japonicum; it is used in traditional medicine for its tonic and fortifying properties, in particular in old age. Numerous effects have been claimed, particularly stimulation of cell immunity and humoral immunity following injection. Use is made generally of root extracts of this plant, which exists in two main types: white ginseng and red ginseng. The roots contain 0.5% to 3% of saponins referred to as ginsenosides (or panaxosides), a complex mixture of dammaran glycosides, many of which have a protopanaxadiol or protopanaxatriol skeleton. The main ginsenosides are written Rb1, Rb2 and Rg1. Ginseng extracts contain other compounds such as sesquiterpenes or alkyne alcohols.

Ginseng extracts particularly suitable for the implementation of the invention are enriched with Rb1 and Rg1 ginsenosides, such as Panax notoginseng extracts. The compositions according to the invention advantageously comprise at least one Rb1 ginsenoside (corresponding to the empirical formula C54H92023) or an Rg1 ginsenoside (of empirical formula C₄₂H₇₂O₄).

What the substances or compounds mentioned have in common is in effect a capacity to stimulate endogenous protectin or protectin precursor synthesis, as has been found, surprisingly, by the applicant in the context of the present invention.

This property has therefore been demonstrated, firstly, by the “c-DNA array” technique, as it is known, which allows transcriptomic targets of the compounds to be identified and the induction of expression of messenger RNAs corresponding to protectin to be evaluated. It is hence possible to identify agents which are active on different skin cells. Further, in exemplary embodiments, active agents can be used in association to improve and/or enhance their efficiency.

Identifying the induction of MRNA synthesis by the test compounds was supplemented by studies by means of specific protectin antibodies, which confirm the increase in the production of the protein, in particular by skin keratinocytes or fibroblasts.

The stimulation of endogenous protectin or protectin precursor synthesis is particularly advantageous since the protective protein is then present in situ, without the need to apply an exogenous protein, which would risk being broken down by the skin enzymes or even rejected by the organism in an immune rejection reaction.

The person skilled in the art is therefore capable of determining other protectin or protectin precursor production activators which are suitable for the implementation of the invention.

The invention likewise provides a method of selecting a product capable of protecting cell membranes, characterized in that it comprises a step which consists in contacting the test product with skin cells and in comparing the proportion of protectin or protectin precursor produced by the said skin cells with the production rate of control skin cells which have not been contacted with the test product.

In particular, the test product will be contacted with a skin cell culture. Non-limitative examples of such cultures are cultures of fibroblasts at confluence or keratinocyte cultures, or reconstructed skin (for example Episkin®).

A comparison is then made of the amount of messenger RNA coding for the protectin synthesized by these cultures with that of a control population, namely a population of cells cultivated under the same conditions with the exception of the test product. If the proportion of mRNA is greater by a factor of at least 1.5, in particular at least two times greater than that of the control culture, the test product is considered to be a protectin or protectin precursor production activator and can be used in accordance with the invention. The synthesis of messenger RNAs is preferably increased by a factor of greater than or equal to 2. The technique is preferably employed with an internal control, such as actin, in order to verify the specificity of the induction.

This first step is advantageously supplemented by tests which show an increase in the level of production of the protein.

The selected product protects, in particular, the cell membranes against the non-specific effects of complement activation. The selection method according to the invention will thus be particularly suitable for selecting more or less complex products having soothing or anti-irritant effects.

According to one advantageous embodiment of the method, it allows the selection of cell extracts enriched in terms of protectin activity, such as, for example, epidermal carpets which can be used as better-tolerated skin grafts. Accordingly, a carpet of epidermal cells is contacted with at least one protectin or protectin precursor activator as defined above for a time suitable for triggering the simulation, and then these pretreated carpets are used for skin grafts which will be tolerated better than standard grafts.

According to yet another aspect, the invention relates to a process in which the cells are stimulated in the presence of various selected extracts which induce protectin or protectin precursors, and then the stimulated cells are used in whole or in part as a source of protectin or protectin precursor for use directly or in a purified form.

The invention therefore likewise provides cells stimulated in vitro by protectin or protectin precursor activators and compositions comprising such stimulated cells or purified extracts obtained from cells thus stimulated or from their culture medium.

The invention also provides for the use of at least one compound selected from bacterial extracts, especially extracts of non-photosynthetic filamentous bacteria, extracts of notoginseng or of ginseng, in particular saponin such as Rb1 and Rg1 ginsenosides, derivatives thereof and mixtures thereof for preparing a composition intended to stimulate the production of protectin or a protectin precursor by skin cells.

The compositions used in accordance with the invention may be present in any form suitable for the intended applications, particularly orally or topically, in the fields of cosmetology and dermatology.

The composition according to the invention may thus be applied to any skin area of the body, the hair, the nails and the mucosae, and may be present in any pharmaceutical form adapted by the person skilled in the art. It may in particular be present in the form of an aqueous or oily suspension or solution, an oil-in-water or water-in-oil emulsion or multiple emulsion, a silicone emulsion, a microemulsion or nanoemulsion, an aqueous or oily gel or a liquid, pastelike or solid anhydrous product.

For topical application to the skin the composition may have the form in particular of an aqueous or oily solution or of a dispersion of the lotion or serum type, emulsions with a liquid or semiliquid consistency of the milk type, obtained by dispersing a fatty phase in an aqueous phase (O/W) or inversely (W/O), or suspensions or emulsions with a soft consistency, of the cream or aqueous or anhydrous gel type, or else microcapsules or microparticles, or vesicle dispersions of ionic and/or non-ionic type. It may also be present in the form of a patch or controlled release system. These compositions are prepared in accordance with the usual methods.

They may also be used for the hair, in the form of aqueous, alcoholic or aqueous-alcoholic solutions, or in the form of creams, gels, emulsions or mousses, or else in the form of aerosol compositions likewise comprising a pressurized propellant.

For the eyes, the composition may be present in the form of drops, and, for ingestion, it may be present in the form of capsules, granules, gels, chewing gums, syrups or tablets.

The amounts of the various constituents of the compositions according to the invention are those conventionally used in the fields in question.

These compositions constitute, in particular, cleansing, protection, treatment or care creams for the face, hands, feet, for the major anatomical folds or for the body (for example day creams, night creams, makeup remover creams, cream foundations, sun protection creams), masks to be left on the skin or hair, liquid foundations, makeup remover lotions, body protection or care lotions, sun protection lotions, lotions, gels or mousses for skincare, such as cleansing lotions, sun protection lotions, artificial tanning lotions, bath compositions, deodorant compositions comprising a bactericide, after-shave gels or lotions, depilatory creams, compositions for treating insect skins, pain relief compositions, and compositions for treating certain diseases of the skin such as eczema, rosacea, psoriasis, lichens or severe pruritus.

The compositions according to the invention may also comprise solid preparations consisting of cleansing bars or soaps.

The compositions may also be packaged in the form of an aerosol composition further comprising a pressurized propellant.

The composition according to the invention may also be a haircare composition, and in particular a shampoo, a hairsetting lotion, a treatment lotion, a styling cream or gel, a dye composition (especially oxidation dyes), where appropriate in the form of coloring shampoos, hair restructuring lotions, a perming composition (in particular a composition for the first step of a perming operation), a lotion or gel for combating hair loss, an antiparasitic shampoo, etc.

The composition may also be for oral use, for example a toothpaste. In this case the composition may comprise adjuvants and additives which are customary for compositions for oral use, and in particular surfactants, thickeners, humectants, polishing agents such as silica, various active ingredients such as fluorides, especially sodium fluoride, and, where appropriate, sweeteners, such as sodium saccharinate.

When the composition is an emulsion the proportion of the fatty phase may range from 5% to 80% by weight, and preferably from 5% to 50% by weight, relative to the total weight of the composition. The oils, waxes, emulsifiers and coemulsifiers used in the composition in emulsion form are selected from those conventionally used in the cosmetics field. The emulsifier and coemulsifier are present in the composition in a proportion ranging from 0.3% to 30% by weight, and preferably from 0.5% to 20% by weight, relative to the total weight of the composition. The emulsion may further comprise lipid vesicles.

When the composition is an oily gel or solution the fatty phase may represent more than 90% of the total weight of the composition.

Conventionally the cosmetic composition may also comprise adjuvants which are common in the cosmetics field, such as hydrophilic or lipophilic gelling agents, hydrophilic or lipophilic additives, preservatives, antioxidants, solvents, fragrances, fillers, filters, odor absorbers and colorants. The amounts of these various adjuvants are those conventionally used in the cosmetic field, and amount for example to from 0.01% to 10% of the total weight of the composition. These adjuvants, depending on their nature, can be introduced in the fatty phase, in the aqueous phase and/or in the lipid spherules.

As oils or waxes which can be used in the invention mention may be made of mineral oils (vaseline oil), vegetable oils (liquid fraction of karite butter, sunflower oil), animal oils (perhydrosqualene), synthetic oils (purcellin oil), silicone oils or waxes (cyclomethicone) and fluoro oils (perfluoropolyethers), beeswax, carnauba wax or paraffin wax. Fatty alcohols and fatty acids (stearic acid) may be added to these oils.

As emulsifiers which can be used in the invention mention may be made, for example, of glyceryl stearate, polysorbate 60 and the PEG-6/PEG-32/glycol stearate mixture sold under the name Tefose®63 by Gattefosse.

As solvents which can be used in the invention mention may be made of lower alcohols, especially ethanol and isopropanol, and propylene glycol.

As hydrophilic gelling agents which can be used in the invention mention may be made of carboxyvinyl polymers (carbomers), acrylic copolymers such as acrylate/alkyl acrylate copolymers, polyacrylamides, polysaccharides such as hydroxy-propylcellulose, natural gums and clays, and, as lipophilic gelling agents, mention may be made of modified clays such as Bentones®, metal salts of fatty acids, such as aluminum stearates, and hydrophobic silica, ethylcellulose and polyethylene.

The amount of protectin or protectin precursor activator present in the compositions according to the invention will be adapted by the person skilled in the art in order to obtain the desired protective activity, depending on the type of activator used. By way of indication, the amount of activator in the compositions will be between 0.001% and 10% by weight, relative to the total weight of the composition, preferably from 0.01% to 5%; in particular it will be at least 0.1%.

The composition may comprise other hydrophilic actives such as proteins or protein hydrolysates, amino acids, polyols, urea, allantoin, sugars and sugar derivatives, water-soluble vitamins, plant extracts and hydroxy acids.

As lipophilic actives it is possible to use retinol (vitamin A) and its derivatives, tocopherol (vitamin E) and its derivatives, essential fatty acids, ceramides, essential oils, salicylic acid and its derivatives.

According to the invention the composition may combine at least one activator of endogenous production of protectin or protectin precursor with other active principles intended in particular for preventing and/or treating skin disorders. Among these active principles mention may be made, by way of example, of:

moisturizers, which may be

either a compound acting on the barrier function, for the purpose of maintaining the hydration of the stratum corneum, or a blocking compound. Mention may be made of ceramides, sphingoid-based compounds, lecithins, glyco-sphingolipids, phospholipids, cholesterol and its derivatives, phytosterols (stigmasterol, β-sitosterol, campesterol), essential fatty acids, 1,2-diacyl-glycerol, 4-chromanone, pentacyclic triterpenes such as ursolic acid, vaseline and lanolin;

or a compound directly increasing the water content of the stratum corneum, such as threalose and its derivatives, hyaluronic acid and its derivatives, glycerol, pentanediol, sodium pidolate, serine, xylitol, sodium lactate, glyceryl polyacrylate, ectoin and its derivatives, chitosan, oligo- and polysaccharides, cyclic carbonates, N-lauroyl-pyrrolidonecarboxylic acid and N-α-benzoyl-L-arginine;

agents which modulate skin differentiation and/or proliferation, such as retinoic acid and its isomers, retinol and its esters, vitamin D and its derivatives; agents stimulating fibroblast proliferation may be selected, for example, from proteins or plant polypeptides, extracts, particularly that of soya (for example a soya extract sold by LSN under the name Eleseryl SH-VEG 8® or sold by SILAB under the trademark Raffermine®); and plant hormones such as gibberellins and cytokinins.

Agents which stimulate the proliferation of keratinocytes and can be used in the composition according to the invention comprise in particular retinoids such as retinol and its esters, including retinyl palmitate; walnut cake extracts sold by Gattefosse; and Solanum tuberosum extracts sold by Sederma.

Agents which stimulate the differentiation of keratinocytes include, for example, minerals such as calcium; lupin extract sold by SILAB under the trademark Photopreventine®; sodium beta-sitosteryl sulphate sold by SEPORGA under the trademark Phytocohesine®; and the maize extract sold by SOLABIA under the trademark Phytovityl®

lycopene, carotenoids and lutein

depigmenting agents such as, for example, the following compounds: kojic acid; ellagic acid; arbutin and its derivatives such as those described in EP-895,779 and EP-524,109; hydroquinone; aminophenol derivatives such as those described in WO 99/10318 and WO 99/32077, and in particular N-cholesteryloxycarbonyl-para-aminophenol and N-ethyloxycarbonyl-para-aminophenol; iminophenol derivatives, especially those described in WO 99/22707; L-2-oxothiazolidine-4-carboxylic acid or procysteine, and its salts and esters; ascorbic acid and its derivatives, especially ascorbyl glucoside; and plant extracts, in particular extracts of liquorice, mulberry and skullcap, without this list being limitative. The composition according to the present invention comprising the aforementioned depigmenting agents is advantageously intended to prevent or treat hyperpigmentation, in particular pigment spots associated with skin ageing.

propigmenting agents: mention may be made of extract of burnet (Sanguisorba officinalis) sold by MARUZEN and chrysanthemum extract (Chrysanthemum morifolium). The composition comprising the aforementioned propigmenting agents is preferably intended to treat canities.

antibacterials such as clindamycin phosphate, erythromycin or antibiotics from the class of the tetracyclines;

antiparasitic agents, especially metronidazole, crotamiton or pyrethrinoids;

antifungals, especially compounds belonging to the class of the imidazoles, such as econazole, ketoconazole or miconazole or their salts, polyene compounds, such as amphotericin B, compounds from the class of the allylamines, such as terbinafine, or else octopirox;

non-steroidal anti-inflammatory agents such as ibuprofen and its salts, diclofenac and its salts, acetylsalicylic acid, acetaminophen or glycyrrhetinic acid;

anti-inflammatories capable of inhibiting the main enzymes involved in the inflammatory process (arachidonic acid cascade), namely: phospholipases A2 (PLA2); lipoxygenases (Lox); and human prostaglandin synthetases (PGHS). Among the raw materials which are effective for inhibiting at least one of these enzymes, mention may be made, without limitation, of the following actives: pentacyclic triterpenes and plant extracts (e.g., Glycyrrhiza glabra) containing them, such as β-glycyrrhetinic acid and its salts and/or its derivatives (glycyrrhetinic acid monoglucuronide, stearyl glycyrrhetinate, 3-stearoyloxy glycyrrhetic acid), ursolic acid and its salts, oleanolic acid and its salts, betulinic acid and its salts, extract of Paeonia suffruticosa and/or lactiflora, calophyllum oil, salts of salicylic acid and especially zinc salicylate, anti-inflammatory phycosaccharides (hydrolysed algin or hydrolysed algin and zinc sulphate) from the company Codif, phlorogine (Laminaria saccharina) from Secma, canola oil, tamanu oil, calophyllum oil, β-bisabolol and camomile extracts, allantoin, Septival EPC (phosphoric diester of vitamin E and C) from Seppic, omega-3 unsaturated oils such as rose musk oil, blackcurrant seed oil, ecchium oil, fish oil, omega plankton (plankton extract) from Secma, lipacide C8G (capryloyl glycine) from Seppic, Seppicalm VG (sodium palmitoylproline and Nymphea alba) from Seppic, extract of rosebay willow herb, extract of pygeum, Soothex (extract of Boswellia serrata) from Quest, phytoplenolin (extract of Centipeda cunninghami) from Bio-Botanica, helioxine (extract of Helianthus annuus) from Silab, Sensiline (Linum usitatissimum) from Silab, tocotrienols, extracts of Cola nitida, piperonal, clove extract, extract of rosebay willow herb (Epilobium angustifolium), aloe vera, bacocalmine (extract of Bacopa moniera) from SEDERMA, phytosterols, cortisone, hydrocortisone, indometacin, beta-methasone and NDGA;

anaesthetics such as lidocaine hydrochloride and its derivatives;

antipruriginous agents such as thenaldine, trimeprazine or cyproheptadine;

keratolytic agents, such as alpha- and beta-hydroxycarboxylic or beta-ketocarboxylic acids, their salts, amides or esters, and more particularly hydroxy acids such as glycolic acid, lactic acid, salicylic acid, citric acid and, generally, fruit acids, and n-octanoyl-5-salicylic acid;

free-radical scavengers, such as vitamin E or alpha-tocopherol, its derivatives or its esters such as tocopheryl acetate, certain metal-chelating agents or ascorbic acid and its esters; bioflavonoids; coenzyme Q10 or ubiquinone; certain enzymes such as catalase, superoxide dismutase, lactoperoxidase, glutathione peroxidase and quinone reductases; glutathione; benzylidenecamphor; benzylcyclanones; substituted naphthalenones; pidolates; phytantriol; gamma-oryzanol; lignans; and melatonin;

antiseborrhoeics, such as progesterone;

agents acting against hair loss, such as aminexil, which is described in EP-540,629, or potassium channel agonists, including minoxidil or 2,4-diamino-6-piperidinopyrimidine and its derivatives, lipoxygenase inhibitors as described in EP-648,488, bradykinin inhibitors, described in particular in EP-845,700, prostaglandins and their derivatives, vasodilators and antiandrogens; -5α-reductase inhibitors selected in particular from retinoids, and especially retinol; sulphur and sulphur derivatives; zinc salts such as zinc lactate, zinc gluconate, zinc pidolate, zinc carboxylate, zinc salicylate and/or zinc cysteate; selenium chloride; vitamin B6 or pyridoxine; the mixture of capryloyl glycine, sarcosine and extract of Cinnamomum zeylanicum sold in particular by SEPPIC under the trademark Sepicontrol A5®; an extract of Laminaria saccharina sold in particular by SECMA under the trademark Phlorogine®; an extract of Spiraea ulmaria sold in particular by SILAB under the trademark Sebonormine®; plant extracts of the species Arnica montana, Cinchona succirubra, Eugenia caryophyllata, Humulus lupulus, Hypericum perforatum, Mentha piperita, Rosmarinus officinalis, Salvia officinalis and Thymus vulgaris, all sold for example by MARUZEN; an extract of Serenoa repens sold in particular by EUROMED; extracts of plants of the genus Silybum; plant extracts containing sapogenins, and in particular the diosgenin-rich or hecogenin-rich Dioscorea extracts; and extracts of Eugenia caryophyllata containing eugenol and eugenyl glucoside. These agents will be present in particular in compositions intended to prevent and/or treat seborrhoea and/or hair loss.

anti-dandruff agents such as octopirox or zinc pyrithione;

antiacne agents such as retinoic acid or benzoyl peroxide;

plant extracts or extracts of microbial origin.

Advantageously the composition according to the invention will comprise nanospheres or microspheres, as described for example in EP-447,318, EP-557,489, EP-1-151,741, EP-1-201,219 or WO 97/12602.

According to one particular embodiment of the invention the compositions comprise at least one protectin or protectin precursor production activator in combination with at least one compound, such as, for example, an active ingredient, known to have a secondary irritant effect.

The irritant products to which the invention applies are, in particular, fragrances, surfactants (ionic or non-ionic), preservatives, certain sunscreens, organic solvents, alcoholic solutions and certain cosmetic, pharmaceutical or dermatological actives.

The products having a secondary irritant effect are selected in particular from α-hydroxy acids (glycolic, lactic, malic, citric, tartaric and mandelic acids), β-hydroxy acids (salicylic acid and its derivatives), α-keto acids, β-keto acids, retinoids (retinol and its esters, retinal, retinoic acid and its derivatives, retinoids, especially those described in FR-A-2,570,377, EP-A-199,636, EP-A-325,540, EP-A-402,072), anthralins (dioxyanthranol), anthranoids, peroxides, minoxidil, lithium salts, antimetabolites, vitamin D and its derivatives, hair dyes or colors (para-phenylenediamine and its derivatives, aminophenols), fragrance-imparting alcoholic solutions (fragrances, toilet waters, after-shaves, deodorants), antiperspirants (certain aluminum salts), depilatory or perming actives (thiols), depigmenting agents (hydroquinone), anti-louse actives (pyrethrine).

The invention further provides a cosmetic method of enhancing the resistance of the skin to the non-specific effects of complement activation, characterized in that at least one protectin or protectin precursor production activator obtainable by the method of selecting described above is applied topically.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLE 1

Measurement of the Increase in Protectin (CD59) Synthesis by Vitreoscilla Filiformis:

I. Apparatus and Methods:

Cell Cultures and Treatment:

The tests were carried out on standard human epidermal keratinocytes in culture.

The keratinocytes were cultivated in SFM medium without pituitary extract and EGF.

The extracts of Vitreoscilla filiformis ATCC1551 obtained in accordance with the protocols described in WO-A-93/00741 were applied to the cells at a concentration of 0.1% (w/v). The contact time was 18 hours.

cDNA Array:

The methodology used is that recommended by Clontech (Palo Alto, USA). The extraction/purification of the total RNA from each culture resulted in the isolation of total RNA quantities of the order of 100 to 150 μg. The total RNA solutions were treated with DNAse I in order to remove any trace of contaminant DNA, in accordance with the supplier's recommendations. The quality of the RNA was then verified on agarose gel and the RNA solutions were adjusted to 1 μg/ml.

The next step was the purification of the messenger RNAs (mRNA) by hybridization of the poly(A) ends of the mRNAs using biotinylated oligo(dT) primers and selective capture on streptavidin beads, in accordance with the Atlaspure protocol (Clontech). ³³P-labeled DNA probes were produced by reverse transcription of the niRNAs bound to poly(dT) beads, using a pool of specific primers of the immobilized sequences on membranes (arrays), in the presence of (α³³P)-dATP. This step used the reagents and the protocol recommended by Clontech. The labeled probes were purified by chromatography on an exclusion column, and the quality and equivalence of the labeled probes was evaluated by liquid scintillation counting.

cDNA array membranes were pretreated and then the cDNAs immobilized on each membrane were hybridized (68° C., overnight) with the corresponding labeled probes. The filters were subsequently washed and placed in individual plastic bags for analysis. Analysis took place by direct quantification of the radioactivity of the spots using a Cyclone phosphoimager (Packard). The results were expressed in relative expression units (RE, radioactivity of the spot corresponding to each gene, corrected for the background noise and for the differences in intensity of labeling of the probes).

RT-Q-PCR:

The primer pairs used in this study were CD59 glycoprotein precursor (CD59, size of the amplified fragment: 321 bp) and actin sequence primers (450 bp). The total RNAs were extracted using Tri-Reagent in accordance with the protocol recommended by the supplier. This was followed by further extraction with chloroform and precipitation from isopropanol. The potentially contaminating traces of DNA were removed by treatment with the DNA-free system (Ambion). The reverse transcriptase reaction was carried out in the presence of the oligo(dT) primer and of the enzyme Superscript II (Gibco). This step was followed by quantification, by fluorescence, of the synthesized cDNA and adjustment of the concentrations to 150 ng/ml. Further quantification of each cDNA, after final dilution, was carried out before the PCR.

PCRs (polymerase chain reactions) were carried out by quantitative PCR using the “Light Cycler” system (Roche Molecular Systems Inc.) and in accordance with the procedures recommended by the supplier. The reaction mixture (10 μl, final) introduced into the capillaries of a thermocycler was composed of 2.5 μl of cDNA, primers of the two labels, the reaction mixture (Roche) containing the enzyme taq DNA polymerase, and the label SYBR Green I. The conditions of the PCR were as follows: 10 min of activation at 95° C., PCR in 40 cycles, melting at 95° C. for 5 sec and then at 60° C. for 5 sec.

Fluorescence analysis in the amplified DNA was measured continuously during the PCR cycles. The mean value of the relative expression (RE) was expressed in Arbitrary Units (AU), calculated from the cycle values of two independent PCRs in accordance with the following formula: (½^{number of cycles})×10⁶.

The results of expression of CD59 were compared with that of the actin, so as to take account of any differences in cell concentration within the two cell populations.

Flow Cytometry Study:

The cells were precultivated at high density for 48 hours and then treated or not with the test product for 24 or 48 hours. The cells were trypsinized and then rinsed with a PBS/2% FCS solution. The cells were transferred to Eppendorf tubes and centrifuged at 1,500 rpm for 5′. The cells were fixed with a PBS-formaldehyde solution at a final concentration of 4%, carried out at ambient temperature and in darkness for 30′. They were subsequently made permeable using a 0.1% Triton-X100/0.1% citrate solution. The cells were labeled in the presence of anti-CD59 antibody (TEBU SOD-110) and anti-human-CD59-FITC (Biosciences 555763) in accordance with the supplier's indications. The analysis of the samples was carried out by cytometry (FACSCAN cytometer, software: Cell Quest; Becton-Dickinson) on the total population. The statistics were carried out on 10,000 cells of each sample. The results were expressed in terms of fluorescence intensity (FI), corresponding to the relative amount of each label per cell in a total population of 10,000 analysed cells.

II. Results:

2.1) cDNA Macroarray Study:

Results on culture of human keratinocytes (signal intensity).

| | | | Comparison |
|---|---|---|---|
| | | | between Control |
| | | Cells Exposed to | Cells and |
| Test Product | Control Cells | the Extract | Exposed Cells (in %) |
| 0.1% extract of | 13.1 | 39.3 | 495 (×4.95) |
| V. filiformis | | | |

It was noted that the cell population, consisting of keratinocytes, exposed to the extract of a non-fruiting non-photosynthetic filamentous bacterium, produces significantly more CD59 mRNA than the control population (approximately 5 times more).

2.2) Q RT-PCR Study:

The results below were those obtained on a human keratinocyte culture.

| | Actin | CD59 | RE* Actin | RE* CD59 | CD59/ | % |
|---|---|---|---|---|---|---|
| Treatment | Cycles | Cycle | (AU) | (AU) | Actin | Control |
| Control | 16.30 | 19.82 | 12.31 | 1.12 | 0.091 | 100 |
| | 16.32 | 19.71 | | | | |
| ViFi | 17.45 | 19.86 | 5.55 | 1.01 | 0.181 | 199 |
| | 17.47 | 19.99 | | | | |

(ViFi: Vitreoscilla filiformis)

RE: relative expression expressed in arbitrary units. These results confirm that the ViFi extract induces CD59 production by a factor of 2.

2.3) Flow Cytometry Study:

Effect of treatment on the relative amount of CD59: the results below were those obtained on a human keratinocyte culture.

Relative amount of CD59 in the control keratinocytes and in the keratinocytes treated with the extract (0.1%, w/v) for 48 hours.

| Treatment | Fluor. Intensity | % |
|---|---|---|
| Control | 114.4 | 100 |
| ViFi Extract | 207.7 | 182 |

(ViFi: Vitreoscilla filiformis)

Under the experimental conditions the test extract showed marked stimulation of expression of the protein CD59 (′1.8).

### EXAMPLE 2

Augmentation of CD59 Synthesis by Ginseng Extracts:

The study was carried out on standard human fibroblasts. Dermal fibroblasts were cultivated in DMEM (Life Technology, ref. 21969035) containing L-glutamine (2 mM, Life Technology, ref. 25030024), 50 IU/ml penicillin and 50 μg/ml streptomycin (Life Technology, ref. 15070063) and 1% foetal calf serum (v/v, Life Technology, ref. 10106151).

A fraction of a root extract of Panax notoginseng (San Qi) containing only the saponins was tested for its activity in accordance with the protocol described in the foregoing example by the technique of cDNA array on fibroblasts. The two principal saponins from the extract were also tested.

The products are as follows:

1—a fraction of an extract of Panax notoginseng (San Qi) containing 6 ginsenosides in the following proportions:

| | |
|---|---|
| Rb1 ginsenoside: | 43% |
| Rb2 ginsenoside: | 1% |
| Rc ginsenoside: | traces |
| Rd ginsenoside: | 13% |
| Re ginsenoside: | 9% |
| Rg1 ginsenoside: | 33% |

2-Rb1 ginsenoside (supplier: Extrasynthese)

3-Rg1 ginsenoside (supplier: Extrasynthese)

All the products were tested at a final concentration of 15 micrograms/ml

The results are reported in the following table:

| | | | Comparision of |
|---|---|---|---|
| | | | the Control |
| | | | Population with |
| | | Fibroblasts | the Population |
| | Control | Exposed | Exposed |
| Test Product | Firoblast (RE) | to the Extract (RE) | to the Extract (in %) |
| San Qi | 6.1 | 13.0 | 214 |
| Rb1 | 6.1 | 13.8 | 277 |
| Rg1 | 6.1 | 16.8 | 277 |

The total extract of ginseng and the ginsenosides Rb1 and Rg1 significantly modify the expression of messenger RNAs coding for the protein CD59.

Ideally, if the aim is to stimulate both the keratinocytes and the fibroblasts of the skin to produce protectin or a protectin precursor, the San Qi or its ginsenosides (acting on fibroblasts) will be combined with the Vitreoscilla filiformis extract (which is more active on the keratinocytes).

### EXAMPLE 3

Formulation for Application to the Scalp:

| Leave-on lotion | | | |
|---|---|---|---|
| Extract of Vitreoscilla filiformis | | 0.2 | g |
| Aminexil | | 1.5 | g |
| Propylene glycol | | 22.8 | g |
| Ethanol 95° | | 55.1 | g |
| Purified water | qs | 100 | g |
| Leave-on lotion | | | |
| Extract of Vitreoscilla filiformis | | 0.1 | g |
| Extract of San Qi | | 1.1 | g |
| Propylene glycol | | 22.8 | g |
| Ethanol 95° | | 55.1 | g |
| Purified water | qs | 100 | g |
| Hair loss prevention lotion | | | |
| Extract of Vitreoscilla filiformis | | 0.1 | g |
| Minoxidil | | 2 | g |
| Propylene glycol | | 22.8 | g |
| Ethanol 95° | | 55.1 | g |
| Purified water | qs | 100 | g |

### EXAMPLE 4

Regenerative cream:

| Extract of Vitreoscilla filiformis from | | |
|---|---|---|
| Example 1 (lyophilized extract) | | 0.1% |
| Extract of San Qi | | 0.05% |
| Carbomer 940 ® (crosslinked polyacrylic acid) | | 0.3% |
| Triethanolamine | | 0.3% |
| Stearic acid | | 3.0% |
| Cetyl alcohol | | 2.0% |
| Self-emulsifiable glyceryl monostearate | | 3.0% |
| Soya oil | | 10.0% |
| Lanolin alcohol | | 2.0% |
| Isopropyl myristate | | 4.0% |
| Cetyl stearyl 2-ethylhexanoate | | 4.0% |
| Perhydrosqualene | | 3.0% |
| Paraffin | | 2.0% |
| Glycerol | | 3.0% |
| Preservatives | | 0.3% |
| Water | qs | 100% |

To prepare this cream the aqueous phase containing glycerol, the preservatives and water was heated to 80° C.; the Carbomer 940 was dispersed therein and was subsequently neutralized with the triethanolamine. The fatty phase, heated and homogenized at 80° C., was introduced with vigorous stirring into the aqueous phase. The extract from the example was dispersed in 10 g of water and introduced at 40° C. into the cream, with stirring. The complete cream was cooled to ambient temperature.

This cream was applied to the skin of the face and neck once or twice daily.

### EXAMPLE 5

Solar Erythema Care Cream (Oil-In-Water Emulsion):

| | | |
|---|---|---|
| Extract from Example 1 (lyophilized extract) | | 0.75% |
| Glyceryl stearate | | 2.00% |
| Polysorbate 60 (Tween 60 sold by ICI) | | 1.00% |
| Stearic acid | | 1.40% |
| Glycyrrhetinic acid | | 2.00% |
| Triethanolamine | | 0.70% |
| Carbomer | | 0.40% |
| Liquid fraction of karite butter | | 12.00% |
| Sunflower oil | | 10.00% |
| Antioxidant | | 0.05% |
| Fragrance | | 0.50% |
| Preservative | | 0.30% |
| Water | qs | 100% |

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A method comprising using at least one protectin (CD59) or protectin precursor production activator in a cosmetic composition or for preparing a composition intended to limit the side-effects of complement activation on the skin, mucosae and/or scalp.

2. The method according to claim 1, wherein the composition is intended for application to the skin, mucosae and/or scalp.

3. The method according to claim 1, wherein the activator or composition is intended to reinforce the resistance of the skin and/or hair follicles.

4. The method according to claim 1, wherein the composition is a soothing and/or anti-irritant composition.

5. The method according to claim 1, wherein the activator or composition is intended to prevent, diminish or treat erythema.

6. The method of claim 5, wherein the erythema is a solar erythema.

7. The method according to claim 1, wherein the activator or composition is intended to treat seborrhoeic skin and acne-affected skin.

8. The method according to claim 1, wherein the activator or composition is intended to treat sensitive skin.

9. The method according to claim 1, wherein the activator or composition is intended to prevent or treat canities.

10. The method according to claim 1, wherein the composition is intended to treat at least one condition selected from the group consisting of herpes, psoriasis, allergy, seborrhoeic dermatitis, a disorder caused by Pityrosporum ovale, urticaria and a maladjustment of the complement activation system.

11. A method comprising using at least one compound selected from the group consisting of a bacterial extract, especially extracts of non-photosynthetic filamentous bacteria, ginseng extracts, especially saponins and ginsenosides, derivatives thereof and mixtures thereof to prepare a composition intended to stimulate the production of protectin or a protectin precursor by skin cells.

12. Cosmetic, dermatocosmetic or dermatological composition comprising at least one protectin or protectin precursor production activator, with the proviso that, if the activator is a total extract of Vitreoscilla filiformis, it is combined with at least one other protectin or protectin precursor activator.

13. Composition according to claim 12, wherein the protectin or protectin precursor production activator is selected from the group consisting of bacterial extracts, especially extracts of non-photosynthetic filamentous bacteria, ginseng extracts, especially saponins and ginsenosides, derivatives thereof and mixtures thereof.

14. Composition according to claim 12, further comprising at least one keratinocyte protectin or protectin precursor production activator in combination with at least one fibroblast protectin or protectin precursor production activator.

15. Composition according to claim 14, wherein the keratinocyte protectin or protectin precursor production activator is an extract of non-photosynthetic filamentous bacteria and the fibroblast protectin or protectin precursor production activator is selected from the group consisting of an extract of Panax ginseng, an extract of Panax notoginseng, an extract of Panax quinquefolium and an extract of Panax japonicus.

16. Composition according to claim 12, wherein the composition is in the form of an aqueous or aqueous-alcoholic lotion or solution, an aqueous, aqueous-alcoholic or oily dispersion or suspension, an oil-in-water emulsion, a water-in-oil emulsion, or a gel of liquid, pastelike, semi-solid or solid consistency.

17. Composition according to claim 12, further comprising microspheres and/or nanospheres.

18. Composition according to claim 12, further comprising at least one active ingredient having an irritant effect.

19. Composition according to claim 18, wherein the active ingredient is selected from the group consisting of an α-hydroxy acid, especially a glycolic, a lactic, a malic, a citric, a tartaric and a mandelic acid, a β-hydroxy acid (salicylic acid and its derivatives), an α-keto acid, a β-keto acid, a retinoid such as retinol and its esters, a retinal, a retinoic acid and its derivatives, an anthralin such as dioxyanthranol, an anthranoid, a peroxide, a minoxidil, a lithium salt, an antimetabolite, a vitamin D and its derivatives, a hair dye or a colorant such as a para-phenylenediamine and its derivatives, an aminophenol, a fragrant alcoholic solution, an antiperspirant, an aluminum salt, a depilatory active, a perming active, a depigmenting agent and an anti-louse active.

20. Method of selecting a product capable of protecting cell membranes, utilizable according to claim 1, the method comprising contacting a test product with skin cells and comparing the proportion of protectin or protectin precursor produced by the skin cells with the production rate of control skin cells which have not been contacted with the test product.

21. Method according to claim 20, further comprising selecting products having anti-irritant effects based on the results of the comparison.

22. Method according to claim 20, further comprising selecting cell extracts enriched in terms of protectin activity, such as, for example, epidermal carpets which can be used as better-tolerated skin grafts.

23. Cosmetic method of enhancing the skin's resistance to the effects of non-specific complement activation, the method comprising topically applying at least one protectin or protectin precursor production activator selected by the method of claim 20.
